(19) United States
(12) Patent Application Publication
(10) Pub. No.: US 2025/0161652 A1
(43) Pub. Date: May 22, 2025

(54) CAP FOR A CONNECTOR OF AN ENTERAL FEEDING KIT, AND FEEDING KIT HAVING A CAP

(71) Applicant: B. Braun Melsungen AG, Melsungen (DE)

(72) Inventors: Andreas Katerkamp, Melsungen (DE); Jan Fragner, Kassel (DE); Eva Seidel, Kassel (DE)

(21) Appl. No.: 18/730,267

(22) PCT Filed: Jan. 12, 2023
(86) PCT No.: PCT/EP2023/050637

§ 371 Date: Jul. 18, 2024

(30) Foreign Application Priority Data
Jan. 19, 2022 (DE) 10 2022 101 151.8

(51) Int. Cl. A61M 39/20

(52) U.S. Cl. CPC A61M 39/20

(57) ABSTRACT

A connector closure cap of a medical fluid-conducting kit, in particular for supplying a nutritional solution, includes a base plate, which surrounds a substantially central through-opening and on a flat side of which a sealing sleeve, which projects substantially perpendicularly to the flat side, is arranged or formed, the internal cross section of which sleeve at least partly overlaps the through-opening in the base plate and is preferably coaxial with the through-opening. The through-opening is closed by a gas-permeable but liquid-impermeable, preferably planar, membrane element. The connector closure cap can be part of a medical fluid-conducting kit.

### CROSS-REFERENCE TO RELATED APPLICATIONS

This application is the United States national stage entry of International Application No. PCT/EP2023/050637, filed on Jan. 12, 2023, and claims priority to German Application No. 2022 101 151.8, filed on Jan. 19, 2022. The contents of International Application No. PCT/EP2023/050637 and German Application No. 10 2022 101 151.8 are incorporated by reference herein in their entireties.

### FIELD

The disclosure relates to a connector closure cap of a medical fluid-conducting set, preferably of the single-use type, or a cap for closing a connector which is connected/connectable to a line for administering a medical fluid, with a base plate surrounding a substantially central through-opening, on one flat side of which a sealing sleeve projecting substantially perpendicularly to the flat side is arranged or formed, the internal cross-section of which overlaps the through-opening in the base plate at least in sections and is preferably coaxial therewith.

### BACKGROUND

Patients who, due to their constitution, can no longer be guaranteed an adequate supply of nutrients via the natural food transport route are usually treated with systems for supplying enteral nutritional solutions. For this purpose, an enteral feeding set (medical fluid-conducting set/hose set) is connected at one end to a container filled with a nutritional solution and at the other end to a patient access. In the enteral feeding set, a connection point for connecting a patient connector to a connector of the patient access is conventionally formed. These connectors are specially designed for enteral feeding, for example in accordance with DIN EN ISO 80369-3. Before administering the nutritional solution or before connecting the patient access connector, it has to be ensured that there are no residual air bubbles in the feeding set. In order to do this, the feeding set is vented and filled with the nutritional solution.

However, enteral nutritional solutions are usually highly viscous, which is why filling and deaerating, also known as priming, of the enteral feeding set takes up a significant proportion of the preparation time, depending on the solution in the range of minutes, of the application. The line has to be completely filled with the nutritional solution and deaerated. Priming is usually stopped manually by the user by actuating a hose clamp as soon as the solution has reached the patient connector or has already flown out of it. This is done according to the application instructions under the observation of the user.

Closable connectors or connectors with closure caps/protective caps/caps for/of systems for administering enteral nutritional solutions are sufficiently well known. For example, a connector for connecting medical fluid containers is disclosed in EP 3 191 166 B1. In the unconnected state, the connector can be closed with a cap. For this purpose, the cap has a sealing plug which engages in an inner lumen of the connector. In order to prevent fluid backflow and to reduce the associated dosing inaccuracies, the connector and the cap have vent openings.

JP 2016-158 656 A, on the other hand, shows a connector with a cap for closing/sealing a fluid path through the connector. The connector is a male connector with a conical surface/cone surface, wherein the cap for sealing the connector has, on the one hand, a conical surface cooperating in a fluid-tight manner with the conical surface of the connector or, on the other hand, a sealing plug projecting into an inner lumen of the connector.

Similarly, a cap for a male connector is disclosed in WO 2018/003 923 A1. The cap has a projection/protrusion which is screwed to a thread of the connector in order to close the connector. In order to seal the connector, the cap also has a sealing plug that engages in an inner lumen of the connector.

Furthermore, WO 2019/243 011 A1 shows a male connector which has a cap that closes a fluid passage of the connector in the unconnected state. In order to connect the cap to the connector, the cap and the connector each have latching elements. For example, the connector may have a recess into which a latching element of the cap engages in the form of a latching projection in order to attach the cap to the connector.

However, all of the known caps are (protective) caps for male connectors with an outer cone (male ENFit connectors). This means that female connectors with an inner cone (female ENFit connectors) and closure caps that are specially formed for enteral feeding, in particular in accordance with DIN EN ISO 80369-3, are not known.

In addition, the known caps have a thread and are screwed onto the connector. This means that these caps have a complex structure due to the additional thread. For sealing, the caps also have conical surfaces that rest against an inner or outer surface of the connector. Secure sealing is not guaranteed via such conical surfaces, which prevents a tight fit of the cap up to a certain fluid pressure as a prerequisite for automatic priming (a PrimeStop function). In addition, the caps are difficult to remove from the connector.

### SUMMARY

The objects and objectives of the disclosure are to eliminate or at least reduce the disadvantages of the prior art and, in particular, to provide a (closure) cap for a (female) connector of an enteral feeding set which enables automatic priming of the enteral feeding set, in particular without supervision by a user.

The disclosure is thus based on the realization that the cap closes the connector in such a way as to prevent the fluid from escaping and at the same time to ensure pressure equalization/air exchange between the inside of the connector and the outside.

The cap is accordingly configured/adapted so that a gas-permeable and liquid-impermeable or hydrophobic and/or oleophobic membrane element is accommodated in the base plate, preferably on a side facing away from the sealing sleeve, in order to cover a through-opening, preferably formed coaxially with the sealing sleeve in the base plate. This membrane element thus acts as a kind of liquid stop, but is at the same time permeable to gases (air). Accordingly, if the line system is primed, i.e. filled with liquid food/nutritional solution, any air contained can then be displaced through the membrane system until the liquid nutritional solution has passed through the line system to the membrane element and is stopped there.

In other words, a connector closure element or a connector closure cap of a medical fluid-conducting set/hose set, in particular for supplying a nutritional solution, has a (frame-like) base plate (closure cap frame) surrounding a substantially central through-opening, on one flat side of which, a (plug-shaped) sealing sleeve projecting substantially perpendicular to the flat side is arranged or formed, wherein the internal cross-section of the sealing sleeve overlaps with the through-opening in the base plate at least in sections and is preferably coaxial therewith. This sealing sleeve is preferably formed to be inserted/pressed into a connector opening in a sealing manner. According to the disclosure, the through-opening in the base plate is sealed with the gas-permeable, but fluid-impermeable (hydrophobic and/or oleophobic), preferably plate-shaped, membrane element.

For this purpose, the base plate may preferably have a radially inward directed (fully circumferential) radial step on/in the through-opening on the circumferential side, for example in the form of an inner ring/ring projection, which further preferably forms a supporting ring surface acting toward the surroundings of the connector (i.e. away from the fluid container), on which the membrane element is preferably placed and possibly fixed.

Further preferably, the radial step (inner ring projection) has a circumferential axial groove which is directly adjacent to the inner circumferential wall of the through-opening and thus forms an axial ring indentation in the supporting ring surface. As a result, the membrane element has a correspondingly shaped axial ring projection (which substantially gives the membrane element the shape of a clamp in cross-section), which engages in the annular circumferential groove of the supporting ring surface (possibly by press fit) when the membrane element is placed on the supporting ring surface.

Further advantageous embodiments are explained below.

In a preferred variant, a circumferential sealing lip may be formed on an outer circumferential surface of the sealing sleeve. Preferably, the sealing lip may protrude in the form of a radius/semicircle/curvature or a triangle from the outer circumferential surface of the sealing sleeve in order to develop the sealing effect locally.

According to an advantageous further development, the sealing lip may protrude 0.03-0.2 mm from the outer circumferential surface of the sealing sleeve in a radial direction of the sealing sleeve.

Furthermore, it may be expedient if the maximum outer diameter of the sealing lip is between 5.6-6.2 mm, preferably between 5.8-6 mm, particularly preferably 5.9 mm.

In a preferred embodiment, a circumferential convex radius may also be formed between the base plate and the sealing sleeve, wherein the circumferential convex radius forms an axial stop when the sealing sleeve is inserted into the fluid passage of the connector. It may be advantageous here if a maximum outer diameter of the sealing lip is spaced from the convex radius by 0.4-1.6 mm, preferably 0.5-0.8 mm, particularly preferably 0.6 mm, in an axial direction of the sealing sleeve.

Furthermore, it may be advantageous if the membrane element is made of an acrylic copolymer or a sintered polymer. Alternatively, another similar material may be used for the membrane element as long as the membrane element allows air or gas exchange and prevents liquids from escaping.

Furthermore, the disclosure relates to a (hose-like/flexible) medical fluid-conducting set/hose set/feeding set for administering a medical fluid with a (hose) line which fluidically connects a container in which the fluid is stored to a, in particular female, connector, preferably according to DIN EN ISO 80369-3, and a cap according to the disclosure described above which closes an internal fluid passage of the connector in fluid connection with the line and in the form of a fluid-tight but air-permeable barrier

It may be advantageous if the connector has a hose/line receiving portion for receiving the (hose) line and a connector receiving portion with an inner cone and a securing means for (fluid-tight) connection to a patient-side, in particular male, connector.

Furthermore, it may be expedient for the connector closure cap to have a sleeve-like sheathing arranged on, in particular molded on, the base plate coaxially to the sealing sleeve and which encloses the sealing sleeve in the radial direction, so that an annular hollow space is formed between the sealing sleeve and the sheathing. The connector receiving portion of the connector can project into this hollow space in order to be pressed by the sheathing radially on the outside against the sealing sleeve radially on the inside. In other words, the sheathing may be undersized with respect to the connector receiving portion, so that the sheathing presses/pushes the connector receiving portion against the sealing sleeve from the radial outside to the radial inside in order to press the sealing surfaces of the connector and the sealing sleeve against each other with a higher contact pressure. In other words, an inner diameter of the sheathing is preferably smaller than or equal to an outer diameter of the connector receiving portion.

In an advantageous further development, at least one crosspiece, preferably extending in the axial direction and projecting in the radial direction, may be formed on an inner circumferential surface of the sheathing. The crosspiece further reduces the inner diameter of the sheathing so that the contact pressure acting on the sealing surfaces of the connector and the sealing sleeve can be further increased.

It may be expedient if the sheathing has a length of 4-8 mm in an axial direction. It may also be advantageous if the sheathing has an internal diameter of 9.8-11 mm.

In other words, the use of a cap according to the disclosure for a connector (also called

‘PrimeStop’ cap) allows automatic priming of the line after starting the filling process without the user having to manually supervise and stop the process (‘PrimeStop’ function). This is because the PrimeStop cap contains a hydrophobic and/or oleophobic membrane (in an outlet channel within the cap connected to the line within the connector), preferably made of an acrylic copolymer, a sintered polymer or similar materials, and thus allows the passage of air while retaining liquids. The PrimeStop cap according to the disclosure is attached to the connector with inner cone by a releasable connection (form-fit and/or force-fit). The connector with inner cone is specially designed for enteral feeding, e.g. in accordance with DIN EN ISO 80369-3. The sealing between the connector with inner cone and the cap is provided by the sealing lip, which is arranged around the outer surface of the sealing sleeve of the cap. The PrimeStop cap is suitable for priming by gravity and by pump. The cap also serves as a protective cap for enteral feeding sets for gravity and pump application. It thus prevents particle contamination of the fluid passage. After successful priming, the cap can be removed from the patient connector and disposed of. The feeding set is then ready for use.

In yet other words, the disclosure relates to the PrimeStop cap comprising the sealing sleeve with the sealing lip circumferentially arranged at the upper end portion of the sealing sleeve. In the present disclosure, the seal is achieved locally via the sealing lip by pressing the sealing lip against an outlet channel-side (distal) portion of the inner surface of the female connector. The sealing lip extends on the outer surface of the sealing sleeve over an (axial) length of 0.4-1.6 mm, preferably 0.5-0.8 mm, particularly preferably 0.6 mm, from the cap-side (axial) stop (contact/stop surface) for the connector defined by the convex radius. The sealing lip protrudes 0.03-0.2 mm in the radial direction from the outer surface/outer circumferential side of the sealing sleeve. The maximum outer diameter/external diameter of the sealing lip is 5.6-6.2 mm, preferably 5.8-6 mm, particularly preferably 5.9 mm.

The sealing lip may advantageously be curved (bead-like) (easier demolding in the mold), but may also be tip-shaped (triangular). In order to support the sealing effect, the cap may have the sleeve-shaped, radially outer sheathing (surrounding the sealing sleeve radially on the outside), through which the additional (radially acting) contact pressure can be applied to the female connector. The sheathing extends over a height (axial length) of 4 to 8 mm.

### BRIEF DESCRIPTION OF THE DRAWINGS

The disclosure is explained in more detail below with reference to preferred configuration examples with the aid of the following Figures, of which:

FIG. 1 shows a schematic structure of an enteral feeding set;

FIG. 2 shows a longitudinal sectional view of a connector with a cap according to a preferred embodiment;

FIG. 3 shows a detailed view of the connector with the cap according to the preferred embodiment;

FIG. 4 shows the detailed view of FIG. 3 without the connector;

FIG. 5 shows a longitudinal sectional view of the connector with a cap according to a first modification of the preferred embodiment; and

FIG. 6 shows a longitudinal sectional view of the connector with a cap according to a second modification of the preferred embodiment.

The Figures are schematic in nature and are provided solely for the purpose of understanding the disclosure. Identical elements are provided with the same reference signs. The features of the various modifications or embodiments may be interchanged.

### DETAILED DESCRIPTION

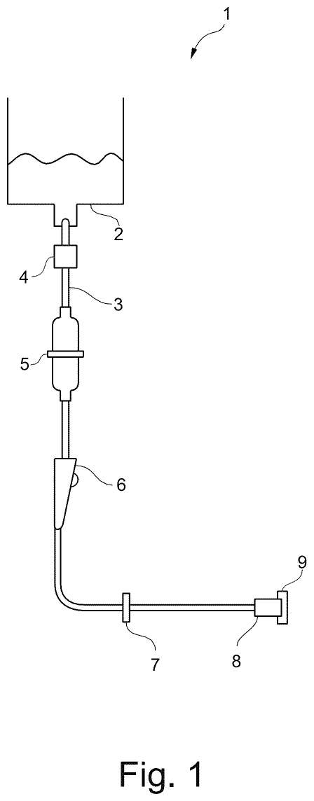

FIG. 1 schematically shows an enteral feeding set 1, i.e. a system for administering an enteral nutritional solution to a patient. For this purpose, the enteral feeding set 1 has a container 2 in which the enteral nutritional solution is stored. A line/hose 3 is connected to this container 2 via a needle connector/cannula 4, so that the enteral nutritional solution can flow into the hose 3 via the needle connector 4.

A drip chamber 5 is arranged in the direction of flow of the enteral nutritional solution downstream of the needle connector 4. Air bubbles, which may be present in the portion of the hose 3 upstream of the drip chamber 5, can be separated in the drip chamber 5. A roller clamp 6 and optionally a hose clamp 7 are provided further downstream of the drip chamber 5. The roller clamp 6 is arranged on the hose 3 in such a way that the roller clamp 6 can press the hose 3 in order to adjust an internal cross-section of the hose 3 and thus a flow rate/volume flow of the enteral nutritional solution. The hose clamp 7 can also press/squeeze the hose 3, wherein the hose clamp 7 can substantially assume a fully open state, in which the hose clamp 7 does not press the hose 3 and the enteral nutritional solution can flow through the hose 3, and a fully closed state, in which the hose clamp 7 presses the hose 3 so that the enteral nutritional solution cannot flow via the hose clamp 7.

In order to be able to connect the hose 3 for administering the enteral nutritional solution to the patient, a connector 8 in the form of a female connector, preferably in accordance with DIN EN ISO 80369-3, is arranged downstream of the hose clamp 7. The connector 8 may be connected to a counterpiece of a patient access (not shown) in a fluid-transmitting manner. The counterpiece is formed in the form of a male connector, preferably in accordance with DIN EN ISO 80369-3.

Before the enteral nutritional solution can be administered to the patient or before the connector 8 of the enteral feeding set 1 can be connected to the counterpiece of the patient access, the tube 3 has to be vented and filled with the enteral nutritional solution (priming). In order to be able to carry out this priming step automatically and without supervision by a user or hospital staff, the connector 8 can be closed with a (closure/protective) cap 9 according to a preferred embodiment. As explained in more detail below, the cap 9 enables fully automatic priming of the hose 3 both by gravity and with the aid of a pump.

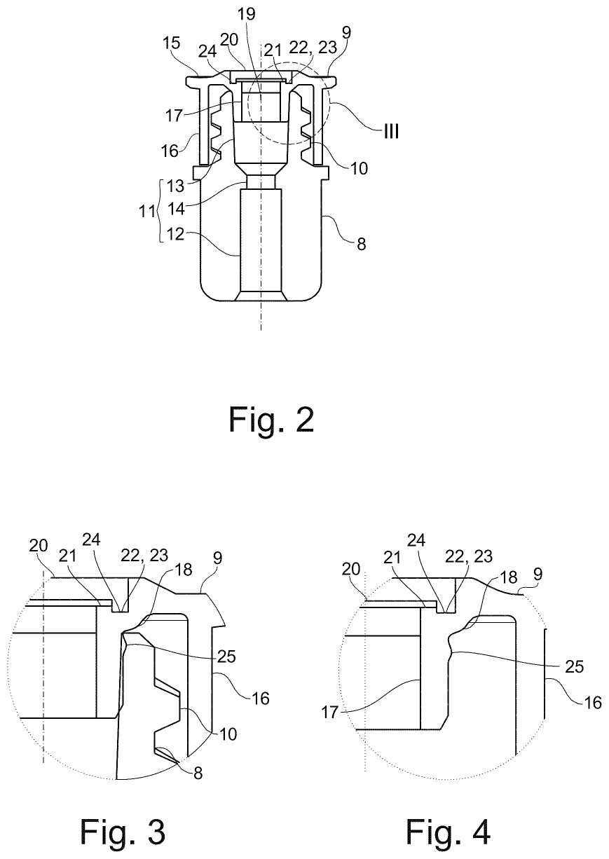

FIG. 2 shows a longitudinal cross-section of the connector 8 and of the cap 9 according to the preferred embodiment. As mentioned above, the connector 8 is designed as a female connector. This means that the connector 8 has an outer thread 10 on an end portion facing the cap 9 (upper end in FIG. 2), which is screwed to an internal thread of the counterpiece when connected to the counterpiece of the patient access. A continuous fluid passage 11 is formed inside the connector 8, which opens outward at both ends of the connector 8.

At an end portion facing away from the cap 9 (lower end in FIG. 2), the connector also has a hose receiving portion 12. The hose 3 can be inserted into the hose receiving portion 12. In other words, the hose receiving portion 12 serves for receiving the hose 3. In the area of the outer thread 10, the fluid passage 11 has a connector receiving portion 13 for receiving the male connector or the counterpiece of the patient access. An inner circumferential surface of the connector receiving portion 13 is designed as an inner cone. A connecting portion 14 is formed between the hose receiving portion 12 and the connector receiving portion 13. As can be seen in FIG. 2, the inner diameters of the hose receiving portion 12 and the connector receiving portion 13 are larger than an inner diameter of the connecting portion 14.

The cap 9 according to the preferred embodiment has a substantially circular base plate 15, from the outer circumferential portion of which an annular outer wall/sheathing 16 extends in the preferred embodiment. A sealing sleeve 17 is formed around the center of the base plate 15, which extends inside the sheathing 16 coaxially to the latter, so that a hollow space is formed between the sealing sleeve 17 and the sheathing 16. As can be seen in FIG. 2, when the cap 9 is connected to the connector 8, the sealing sleeve 17 engages in the fluid passage 11, in particular in the connector receiving portion 13. A convex radius 18 is formed between the sealing sleeve 17 and the base plate 15, which serves as a stop when the cap 9 is pushed onto the connector 8. In other words, the cap 9 is pushed onto the connector 8 or the sealing sleeve 17 is pushed into the connector receiving portion 13 until the radius 18, in particular a portion of the radius 18 facing the connector 8, rests against an end face of the connector 8.

The sealing sleeve 17 furthermore has a through-opening 19, which extends centrally/coaxially through the sealing sleeve 17 and the base plate 15. An opening of the through-opening 19 facing away from the connector 8 is covered with a membrane element 20 in the cap 9 according to the preferred embodiment. The membrane element 20 is accommodated in the base plate 15 of the cap 9 in order to cover the opening of the through-opening 19 facing away from the connector 8.

For this purpose, the base plate 15 has a circumferentially inward directed, fully circumferential radial step 21 on/in the through-opening 19 according to the first embodiment. As can be seen in FIG. 2, the radial step 21 forms a supporting ring surface 22 acting toward the surroundings of the connector 8 (i.e. away from the fluid container or the hose receiving portion 12), on which the membrane element 20 is placed and fixed.

Furthermore, the radial step 21 has a circumferential axial groove 23, which is directly adjacent to the inner circumferential wall of the through-opening 19 and thus forms an axial ring indentation in the supporting ring surface 22. As shown in FIG. 2, the membrane element 20 has a correspondingly shaped axial ring projection 24. In cross-section, the membrane element 20 thus substantially has the shape of a clamp which, when the membrane element 20 is placed on the supporting ring surface 22, engages in the circumferential axial groove 23 thereof and is held in the latter via an interference fit.

In the preferred embodiment, the membrane element 20 is produced as a hydrophobic and/or oleophobic membrane. In particular, the membrane element 20 may be made of an acrylic copolymer or a sintered polymer. Of course, the membrane element 20 may also be made of any other material, as long as the membrane element 20 enables air or gas exchange between an inner side and an outer side of the cap 9 and at the same time prevents liquids from escaping.

As shown in FIGS. 3 and 4, a circumferential sealing lip 25 is formed on an outer circumferential surface of the sealing sleeve 17. In the preferred embodiment, the sealing lip 25 is formed as a curvature/radius, i.e. with a roundish cross-section. Alternatively, however, the sealing lip 25 can also have a tip-shaped/triangular cross-section.

In the preferred embodiment, a maximum outer diameter Dmax of the sealing lip 25 or a maximum radial extension is 5.6-6.2 mm, preferably 5.8-6 mm, particularly preferably 5.9 mm. The sealing lip 25 therefore protrudes in the radial direction from the outer circumferential surface of the sealing sleeve 17 by approximately 0.03-0.2 mm. The maximum outer diameter Dmax of the sealing lip 25 is spaced from the radius 18, as shown in FIG. 4. A distance h between the radius 18 and the maximum outer diameter Dmax is 0.4-1.6 mm, preferably 0.5-0.8 mm, particularly preferably 0.6 mm.

When the cap 9 is now placed on the connector 8, the sealing lip 25 of the sealing sleeve 17 is pressed against an inner circumferential surface of the connector receiving portion 13. This means that the seal between the connector 8 and the cap 9 is achieved locally by the sealing lip 25 and not over the entire inner circumferential surface of the connector receiving portion 13 and the entire outer circumferential surface of the sealing sleeve 17.

In order to increase the contact pressure between the sealing sleeve 17 and the connector receiving portion 13, the cap 9 has the sheathing 16 according to the preferred embodiment. The sheathing 16 is in direct contact with the outer thread 10 of the connector 8. This means that the sealing sleeve 17 and the sheathing 16 are braced against the connector 8. The sheathing 16 may extend over the entire axial length of the outer thread 10, so that the axial length of the sheathing 16 is preferably between 4-8 mm. In the preferred embodiment, the sheathing 16 has an inner diameter of 9.8-11 mm.

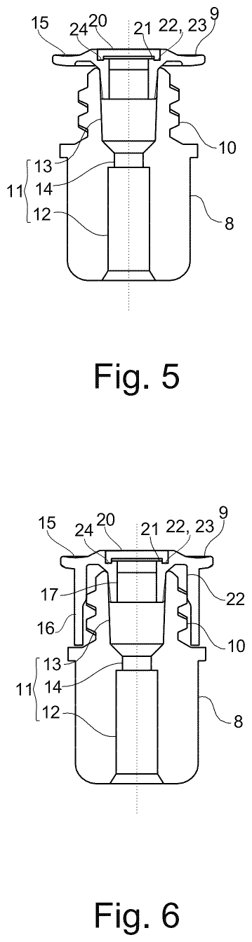

FIG. 5 shows a longitudinal section of the connector 8 and the cap 9 according to a first modification of the preferred embodiment. Here, the cap 9 is designed without the sheathing 16. In other words, the contact pressure is exclusively caused by the oversize of the sealing lip 25 in relation to the inside diameter of the connector receiving portion 13.

In the cap 9 shown in FIG. 6 according to a second modification of the preferred embodiment, at least one crosspiece 26 is formed on the inner circumferential surface of the sheathing 16. The at least one crosspiece 26 is formed at the level of the sealing lip 25 and is in direct contact with the outer thread 10 of the connector 8. As a result, the at least one crosspiece 26 enables an additional increase in the contact pressure.

### List of reference signs

1 feeding set
2 container
3 hose
4 needle connector
5 drip chamber
6 roller clamp
7 hose clamp
8 connector
9 cap
10 outer thread
11 fluid passage
12 hose receiving portion
13 connector receiving portion
14 connecting portion
15 base plate
16 sheathing
17 sealing sleeve
18 convex radius
19 through-opening
20 membrane element
21 radial step
22 supporting ring surface
23 axial groove
24 ring projection
25 sealing lip
26 crosspiece

1-10. (canceled)

11. A connector closure cap of a medical fluid-conducting set, the connector closure cap comprising:
a base plate surrounding a through-opening, the through-opening having a flat side comprising a sealing sleeve that projects from the flat side, wherein an internal cross-section of the sealing sleeve overlaps with the through-opening in the base plate at least in sections,
the through-opening in the base plate being sealed with a membrane element that is gas-permeable but liquid-impermeable,
the base plate having a radial step that is radially-inwardly directed on/in the through-opening on a circumferential side, the radial step forming a supporting ring surface for the membrane element,
the radial step comprising a circumferential axial groove adjacent to an inner circumferential wall of the through-opening, and
the membrane element having an axial ring projection having a shape that corresponds to a geometry of the circumferential axial groove, the axial ring projection configured to engage in the circumferential axial groove when the membrane element is placed on the supporting ring surface.

12. The connector closure cap according to claim 11, wherein a circumferential sealing lip is formed on an outer circumferential surface of the sealing sleeve.

13. The connector closure cap according to claim 11, wherein a circumferential convex radius is formed between the base plate and the sealing sleeve, wherein the circumferential convex radius forms an axial stop when the sealing sleeve is inserted into a fluid passage of a connector.

14. The connector closure cap according to claim 11, wherein the membrane element is made of an acrylic copolymer or a sintered polymer.

15. A medical fluid-conducting set for administering a medical fluid stored in
a container, the medical fluid-conducting set comprising:
the connector closure cap according to claim 11; and
a line connectable to the container for fluidically connecting the container to a connector, wherein the connector closure cap closes an internal fluid passage of the connector when the connector is in fluid communication with the line, forming a fluid-tight and air-permeable barrier.

16. The medical fluid-conducting set according to claim 15, further comprising a connector having a hose receiving portion for receiving the line and a connector receiving portion with an inner cone and a securing element for fluid-tight connection to a patient-side connector.

17. The medical fluid-conducting set according to claim 16, wherein the connector closure cap has a sheathing arranged on the base plate coaxially to the sealing sleeve, the sheathing enclosing the sealing sleeve in a radial direction so that a hollow space is formed between the sealing sleeve and the sheathing, the connector receiving portion projecting into the hollow space in order to be pressed against the sealing sleeve by the sheathing.

18. The medical fluid-conducting set according to claim 17, further comprising at least one crosspiece formed on an inner circumferential surface of the sheathing.
